(19) United States
(12) Patent Application Publication
(10) Pub. No.: US 2019/0231411 A1
(43) Pub. Date: Aug. 1, 2019

(54) METHOD FOR PREDICTING CYTOTOXICITY OF COLD ATMOSPHERIC PLASMA TREATMENT ON CANCER CELLS

(71) Applicant: U.S. Patent Innovations, LLC, Takoma Park, MD (US)

(72) Inventors: Jerome Canady, Lakeland, FL (US); Michael Keidar, Washington, DC (US)

(21) Appl. No.: 16/259,923

(22) Filed: Jan. 28, 2019

Related U.S. Application Data
(60) Provisional application No. 62/622,210, filed on Jan. 26, 2018.

(51) Int. Cl. A61B 18/04; A61N 1/44; G16H 20/40; C12N 5/00; G16H 40/60

(52) U.S. Cl. CPC A61B 18/042; A61N 1/44; A61B 2018/00696; C12N 5/0093; G16H 40/60; G16H 20/40

(57) ABSTRACT

A method for applying cold atmospheric plasma treatment to target tissue comprising the steps of selecting through a graphical user interface on a computing device a particular cancer cell line associated with said target tissue, retrieving, with said computing device, settings data from a database of cell line data and associated settings data in a storage, applying, with said computing device, said retrieved settings data to a cold atmospheric plasma system, and treating cancer tissue with cold atmospheric plasma at the retrieved settings.

### CROSS-REFERENCE TO RELATED APPLICATIONS

The present application claims the benefit of the filing date of U.S. Provisional Patent Application Ser. No. 62/622,210 filed on Jan. 26, 2018.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to systems and methods for treating cancerous tissue with cold atmospheric plasma.

### Brief Description of the Related Art

The unique chemical and physical properties of cold atmospheric plasmas enable their numerous recent applications in biomedicine including sterilization, the preparation of polymer materials for medical procedures, wound healing, tissue or cellular removal and dental drills. A. Fridman, Plasma Chemistry (Cambridge University Press, 2008); G. Fridman, G. Friedman, A. Gutsol, A. B. Shekhter, V. N. Vasilets, and A. Fridman “Applied Plasma Medicine”, Plasma Processes Polym. 5, 503 (2008); E. Stoffels, Y. Sakiyama, and D. B. Graves “Cold Atmospheric Plasma: Charged Species and Their Interactions With Cells and Tissues” IEEE Trans. Plasma Sci. 36, 1441 (2008); X. Lu, Y. Cao, P. Yang, Q. Xiong, Z. Xiong, Y. Xian, and Y. Pan “An RC Plasma Device for Sterilization of Root Canal of Teeth” IEEE Trans. Plasma Sci. 37, 668 (2009).

Plasma-based nitrogen oxide (NO) therapy demonstrated huge potential for stimulation of regenerative processes and wound healing. The work uncovering function of nitrogen oxide as a signal molecule was awarded by the Nobel Prize in medicine and biology in 1999. NO-therapy demonstrated tremendous effect of acceleration of healing of ulcer, burns and serious wounds. Other experimental evidence supports efficiency of cold plasmas produced by dielectric barrier discharge for apoptosis of melanoma cancer cell lines, treatment of cutaneous leishmaniasis, ulcerous eyelid wounds, corneal infections, sterilization of dental cavities, skin regeneration, etc.

Recent progress in atmospheric plasmas led to creation of cold plasmas with ion temperatures close to room temperature. Cold non-thermal atmospheric plasmas can have tremendous applications in biomedical technology. K. H. Becker, K. H. Shoenbach and J. G. Eden “Microplasma and applications” J. Phys. D.:Appl. Phys. 39, R55-R70 (2006). In particular, plasma treatment can potentially offer a minimum-invasive surgery that allows specific cell removal without influencing the whole tissue. Conventional laser surgery is based on thermal interaction and leads to accidental cell death i.e. necrosis and may cause permanent tissue damage. In contrast, non-thermal plasma interaction with tissue may allow specific cell removal without necrosis. In particular, these interactions include cell detachment without affecting cell viability, controllable cell death etc. It can be used also for cosmetic methods of regenerating the reticular architecture of the dermis. The aim of plasma interaction with tissue is not to denaturate the tissue but rather to operate under the threshold of thermal damage and to induce chemically specific response or modification. In particular presence of the plasma can promote chemical reaction that would have desired effect. Chemical reaction can be promoted by tuning the pressure, gas composition and energy. Thus the important issues are to find conditions that produce effect on tissue without thermal treatment. Overall plasma treatment offers the advantage that is can never be thought of in most advanced laser surgery. E. Stoffels, I. E Kieft, R. E. J Sladek, L. J. M van den Bedem, E. P van der Laan, M. Steinbuch “Plasma needle for in vivo medical treatment: recent developments and perspectives” Plasma Sources Sci. Technol. 15, S169-S180 (2006).

Several different systems and methods for performing Cold Atmospheric Plasma (CAP) treatment have been disclosed. For example, U.S. Published Patent Application No. 2014/0378892 discloses a two-electrode system for CAP treatement. U.S. Pat. No. 9,999,462 discloses a converter unit for using a traditional electrosurgical system with a single electrode CAP accessory to perform CAP treatment.

As a near-room temperature ionized gas, cold atmospheric plasma (CAP) has demonstrated its promising capability in cancer treatment by causing the selective death of cancer cells in vitro. See, Yan D, Sherman J H and Keidar M, “Cold atmospheric plasma, a novel promising anti-cancer treatment modality,” Oncotarget. 8 15977-15995 (2017); Keidar M, “Plasma for cancer treatment,” Plasma Sources Sci. Technol. 24 33001 (2015); Hirst A M, Frame F M, Arya M, Maitland N J and O'Connell D, “Low temperature plasmas as emerging cancer therapeutics: the state of play and thoughts for the future,” Tumor Biol. 37 7021-7031 (2016). The CAP treatment on several subcutaneous xenograft tumors and melanoma in mice has also demonstrated its potential clinical application. See, Keidar M, Walk R, Shashurin A, Srinivasan P, Sandler A, Dasgupta S, Ravi R, Guerrero-Preston R and Trink B, “Cold plasma selectivity and the possibility of a paradigm shift in cancer therapy,” Br. J. Cancer. 105 1295-301 (2011); Vandamme M, Robert E, Dozias S, Sobilo J, Lerondel S, Le Pape A and Pouvesle J-M, “Response of human glioma U87 xenografted on mice to non thermal plasma treatment,” Plasma Med. 1 27-43 (2011); Brulle L, Vandamme M, Ries D, Martel E, Robert E, Lerondel S, Trichet V, Richard S, Pouvesle J M and Le Pape A, “Effects of a Non thermal plasma treatment alone or in combination with gemcitabine in a MIA PaCa2-luc orthotopic pancreatic carcinoma model,” PLoS One. 7 e52653 (2012); and Chernets N, Kurpad D S, Alexeev V, Rodrigues D B and Freeman T A, “Reaction chemistry generated by nanosecond pulsed dielectric barrier discharge treatment is responsible for the tumor eradication in the B16 melanoma mouse model,” Plasma Process. Polym. 12 1400-1409 (2015).

The rise of intracellular reactive oxygen species (ROS), DNA damage, mitochondrial damage, as well as apoptosis have been extensively observed in the CAP-treated cancer cell lines. See, Ahn H J, Kim K Il, Kim G, Moon E, Yang S S and Lee J S, “Atmospheric-pressure plasma jet induces apoptosis involving mitochondria via generation of free radicals,”. PLoS One. 6 e28154 (2011); Ja Kim S, Min Joh H and Chung T H, “Production of intracellular reactive oxygen species and change of cell viability induced by atmospheric pressure plasma in normal and cancer cells,” Appl. Phys. Lett. 103 153705 (2013); and Yan D, Talbot A, Nourmohammadi N, Sherman J H, Cheng X and Keidar M, “Toward understanding the selective anticancer capacity of cold atmospheric plasma—a model based on aquaporins (Review),” Biointerphases. 10 040801 (2015). The increase of intracellular ROS may be due to the complicated intracellular pathways or the diffusion of extracellular ROS through the cellular membrane. See, Yan D, Xiao H, Zhu W, Nourmohammadi N, Zhang L G, Bian K and Keidar M, “The role of aquaporins in the anti-glioblastoma capacity of the cold plasma-stimulated medium,” J. Phys. D. Appl. Phys. 50 055401 (2017). However, the exact underlying mechanism is still far from clear.

Cancer cells have shown specific vulnerabilities to CAP. See, Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015)

Understanding the vulnerability of cancer cells to CAP will provide key guidelines for its application in cancer treatment. Only two general trends about the cancer cells' vulnerability to CAP treatment have been observed in vitro based on just a few cell lines. First, one study just compared the cytotoxicity of CAP treatment on the cancer cell lines expressing p53 with the same treatment on the cancer cell lines without expressing p53. The cancer cells expressing the p53 gene were shown to be more resistant to CAP treatment than p53 minus cancer cells. Ma Y, Ha C S, Hwang S W, Lee H J, Kim G C, Lee K W and Song K, “Non-thermal atmospheric pressure plasma preferentially induces apoptosis in p53-mutated cancer cells by activating ROS stress-response pathways,” PLoS One. 9 e91947 (2014). p53, a key tumor suppressor gene, not only restricts abnormal cells via the induction of growth arrest or apoptosis, but also protects the genome from the oxidative damage of ROS such as H₂O₂ through regulating the intracellular redox state. Sablina A A, Budanov A V, Ilyinskaya G V, Larissa S, Kravchenko J E and Chumakov P M, “The antioxidant function of the p53 tumor suppressor,” Nat. Med. 11 1306 (2005). p53 is an upstream regulator of the expression of many anti-oxidant enzymes such as glutathione peroxidase (GPX), glutaredoxin 3 (Grx3), and manganese superoxide dismutase (MnSOD). Maillet A and Pervaiz S, “Redox regulation of p53, redox effectors regulated by p53: a subtle balance,” Antioxid. Redox Signal. 16 1285-1294 (2012). In addition, the cancer cells with a lower proliferation rate are more resistant to CAP than cancer cells with a higher proliferation rate. Naciri M, Dowling D and Al-Rubeai M, “Differential sensitivity of mammalian cell lines to non-thermal atmospheric plasma,” Plasma Process. Polym. 11 391-400 (2014). This trend may be due to the general observation that the loss of p53 is a key step during tumorigenesis. Tumors at a high tumorigenic stage are more likely to have lost p53. See, Fearon E F and Vogelstein B, “A genetic model for colorectal tumorigenesis,” Cell. 61 759-767 (1990).

Despite the complicated interaction between CAP and cancer cells, the initial several hours after treatment has been found to be an important stage for the cytotoxicity of CAP. The anti-cancer ROS molecules in the extracellular medium are completely consumed by cells during this time period. After the initial several hours, replacing the medium surrounding the cancer cells does not change the cytotoxicity of CAP. See, Yan D, Cui H, Zhu W, Nourmohammadi N, Milberg J, Zhang L G, Sherman J H and Keidar M, “The specific vulnerabilities of cancer cells to the cold atmospheric plasma-stimulated solutions,” Sci. Rep. 7 4479 (2017).

### SUMMARY OF THE INVENTION

Cold atmospheric plasma (CAP) has shown its promising capability in cancer treatment both in vitro and in vivo. However, the anti-cancer mechanism is still largely unknown. CAP may kill cancer cells via triggering the rise of intracellular reactive oxygen species (ROS), DNA damage, mitochondrial damage, or cellular membrane damage. While the specific vulnerability of cancer cells to CAP has been observed, the underlying mechanism of such cell-based specific vulnerability to CAP is not yet known. Through the comparison of CAP treatment and H₂O₂ treatment on 10 different cancer cell lines in vitro, we observed that the H₂O₂ consumption rate by cancer cells was strongly correlated to the cytotoxicity of CAP treatment on cancer cells. Cancer cells that clear extracellular H₂O₂ more quickly are more resistant to the cytotoxicity of CAP treatment. This finding strongly indicates that the anti-oxidant system in cancer cells play a key role in the specific vulnerability of cancer cells to CAP treatment in vitro.

The H₂O₂ consumption rate of cancer cells serves as an important cellular physiological marker to predict the cytotoxicity of CAP treatment of cancer cell lines in vitro. The cancer cells which can clear the extracellular H₂O₂ at a faster rate tend to show stronger resistance to CAP treatment or H₂O₂ treatment. This trend first provides a simple method to predict the vulnerability of cancer cells to CAP by monitoring the evolution of H₂O₂ during the initial several hours post the treatment.

Various cancer cell line can be tested in this manner to provide a rough prediction of which cells lines are susceptible to treatment with CAP and further, the various cancer cell lines can be tested at varying settings to provide an estimate of which CAP treatment settings or dosages will provide the greatest affect on particular cancer cell lines. The results of this testing may be used to generate a database of cancel cell lines with associated predicted optimum setting or dosage data and optionally effectiveness data. This database can be stored in memory or other storage in a CAP capable electrosurgical system or can be in an external storage that can be accessed by an CAP capable electrosurgical system. The CAP capable electrosurgical system may have a user interface that then allows a user to enter an identifier for a particular cancer cell line into the user interface and thereby have the CAP enabled electrosurgical system automatically select the predicted optimum settings or dosage for that particular cancer cell line. The user can then perform a CAP treatment of target cancer cells at those predicted optimum settings.

In a preferred embodiment the method for applying cold atmospheric plasma treatment to target tissue comprises the steps of selecting through a graphical user interface on a computing device a particular cancer cell line associated with the target tissue, retrieving, with the computing device, settings data from a database of cell line data and associated settings data in a storage, applying, with the computing device, the retrieved settings data to a cold atmospheric plasma system, and treating cancer tissue with cold atmospheric plasma at the retrieved settings.

In another preferred embodiment, the method for applying cold atmospheric plasma treatment to target tissue comprises generating a database of cancer cell lines and associated cold atmospheric plasma settings, storing the database in a storage medium, selecting through a graphical user interface on a computing device a particular cancer cell line associated with the target tissue, retrieving, with the computing device, settings data from a database of cell line data and associated settings data in a storage; and applying, with the computing device, the retrieved settings data to a cold atmospheric plasma system. In one embodiment of the invention, the cold atmospheric plasma settings in the generated database are based upon a predicted CAP effectiveness derived an H₂O₂ consumption rate of cancer cells in a particular cancer cell line after CAP treatment. In another embodiment of the invention, the cold atmospheric plasma settings in the generated database are based upon a predicted CAP effectiveness derived predicting cytotoxicity of cold atmospheric plasma treatment on particular cancer cell lines.

In one embodiment of the invention, said step of generating a database comprises removing all medium used to culture a plurality of samples of a first cancer cell line, adding DMEM or RPMI to each of said plurality of samples of said first cancel cell line, treating each of said plurality of samples of said first cancer cell line with direct CAP treatment, wherein each of said plurality of samples of said first cancer cell line is treated using only one of a plurality of specific sets of CAP settings and wherein at least two of said plurality of samples of said first cancer cell line are treated with different specific sets of CAP settings, adding a pre-determined amount of H₂O₂-containing medium to each of plurality of samples of said first cancer cell line, culturing said plurality of samples of said first cancer cell line for a pre-determined period of time under pre-determined conditions, measuring an H₂O₂ consumption rate by cancer cells in each treated sample of said first cancer cell line; and predicting optimum CAP settings for said first cancer cell line using obtained measurements of H₂O₂ consumption rate by cancer cells in each treated sample of said first cancer cell line.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1A is a perspective view of a preferred embodiment of a gas-enhanced electrosurgical generator.

FIG. 1B is a front view of a preferred embodiment of a gas-enhanced electrosurgical generator.

FIG. 1C is a rear view of a preferred embodiment of a gas-enhanced electrosurgical generator.

FIG. 1D is a left side view of a preferred embodiment of a gas-enhanced electrosurgical generator.

FIG. 1E is a right view of a preferred embodiment of a gas-enhanced electrosurgical generator.

FIG. 1F is a top view of a preferred embodiment of a gas-enhanced electrosurgical generator.

FIG. 1G is a bottom view of a preferred embodiment of a gas-enhanced electrosurgical generator.

FIG. 2A is a block diagram of a preferred embodiment of pressure control system of a gas-enhanced electrosurgical generator in accordance with the present invention configured to perform an argon-enhanced electrosurgical procedure.

FIG. 2B is a block diagram of a preferred embodiment of pressure control system of a gas-enhanced electrosurgical generator in accordance with the present invention configured to perform a cold atmospheric plasma procedure.

FIG. 2C is a diagram of a trocar for the embodiment of FIG. 2A in accordance with the present invention.

FIG. 2D is a block diagram of an alternate preferred embodiment of pressure control system of a gas-enhanced electrosurgical generator in accordance with the present invention configured to perform an argon-enhanced electrosurgical procedure.

FIG. 3A is a schematic flow diagram illustrating the gas flow through the module and the method by which the module controls the gas flow in accordance with a preferred embodiment of the present invention.

FIG. 3B is a schematic flow diagram illustrating the gas flow through an alternate embodiment of the module and the method by which the module controls the gas flow in accordance with a preferred embodiment of the present invention.

FIG. 4 is a diagram of a graphical user interface in accordance with a preferred embodiment of the present invention.

FIG. 5A is a diagram of an experimental setup used in experiments performed in connection with the present invention.

FIG. 5B illustrates a 12.5 kHz alternating current voltage (3 kV) use in an experimental setup used in experiments performed in connection with the present invention.

FIG. 5C is a graph illustrating the optical emission spectrum (OES) of the plasma jet 20 mm to the glass tube nozzle and 5 mm to the jet axis.

FIGS. 6A and 6B are graphs illustrating the effect of CAP treatment on 10 cancer cell lines.

FIGS. 7A and 7B are graphs illustrating the effect of H₂O₂ treatment on 10 cancer cell lines.

FIGS. 8A and 8B are graphs illustrating the inversely proportional correlation between the H₂O₂ consumption rates of cancer cells and the growth inhibition effect of CAP treatment or H₂O₂ treatment on cancer cells. (a) CAP treatment. (b) H₂O₂ treatment.

FIG. 9 is a flow chart of a method for performing CAP treatment in accordance with a preferred embodiment of the present invention.

### DETAILED DESCRIPTION OF THE PREFERRED EMBODIMENTS

As shown in the experiments discussed below, various cancer cell line can be tested to provide a rough prediction of which cells lines are susceptible to treatment with CAP and further, the various cancer cell lines can be tested at varying settings or dosages of the CAP treatment to provide an estimate of which CAP treatment settings or dosages will provide the greatest effect on particular cancer cell lines. In a preferred embodiment of the present invention, the results of such testing are used to generate a database of cancer cell lines with associated predicted optimum settings or dosage data and optionally effectiveness data. This database can be stored in memory or other storage in a CAP capable electrosurgical system or can be in an external storage, for example, accessible through a server or cloud computing system, that can be accessed by a CAP capable electrosurgical system. The CAP capable electrosurgical system may have a graphical user interface that allows a user to enter an identifier for a particular cancer cell line into the user interface and thereby have the CAP enabled electrosurgical system automatically select the predicted optimum settings or dosage for that particular cancer cell line. The user can then perform a CAP treatment of target cancer cells at those predicted optimum settings.

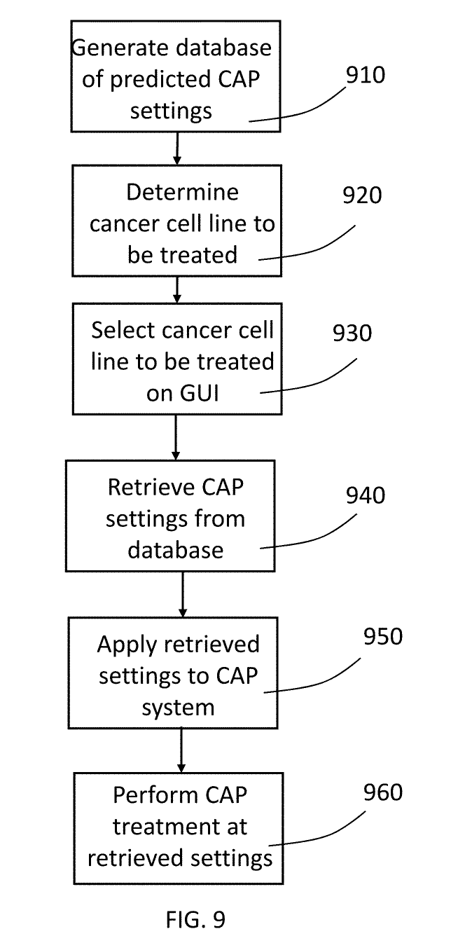

Thus, as shown in FIG. 9, a method can be performed in which a database of cancer cell lines and associated CAP treatment data is generated and stored (step 910). Additional cancer cell line data and associate settings or dosage data can be added to the database as new cell lines are tested and new data is developed. A user performing a CAP treatment determines the particular cancer cell line to be treated (step 920) and then enters an identifier associated with a particular cancer cell line into a graphical user interface on a CAP capable electrosurgical system (step 930). The CAP capable electrosurgical system then accesses the stored database to retrieve CAP setting or dosages associated with the ID entered into the graphical user interface (step 940). The phrase “enter an identifier” used herein can mean any data entry or selection by the user that provides the graphical user interface with sufficient information to retrieve data from the database for a particular cell line. This could be selection from a list or menu, entry of an identifier through a physical or virtual keyboard associated with the system, scanning of a bar code, or any other means. Further, the graphical user interface and associated display do not need to physically be in the CAP capable generator but instead may be on external devices such as a tablet computing device that is in communication with the CAP enabled electrosurgical generator. The retrieved CAP settings are then applied to the CAP system (step 950). The user then can treat the target tissue with CAP at the preferred settings (step 960).

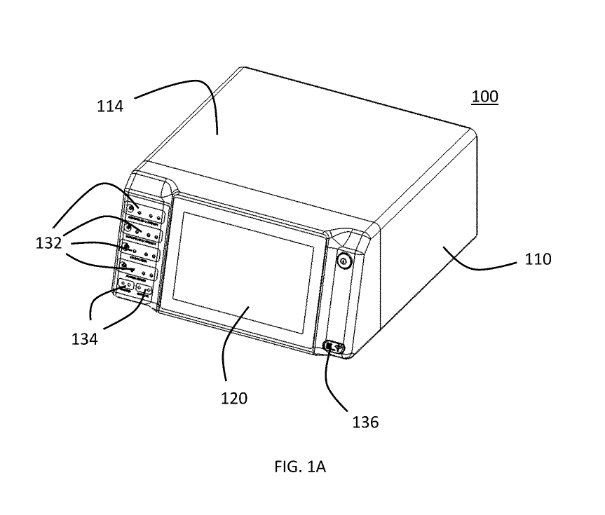

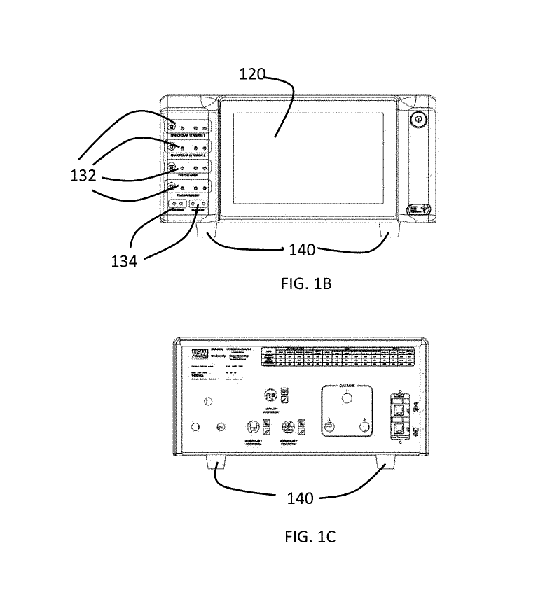

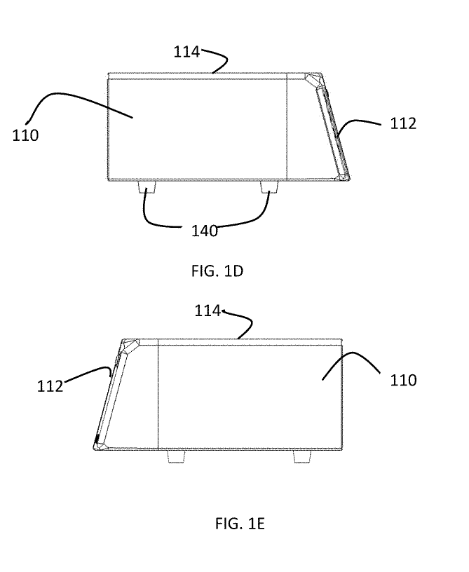

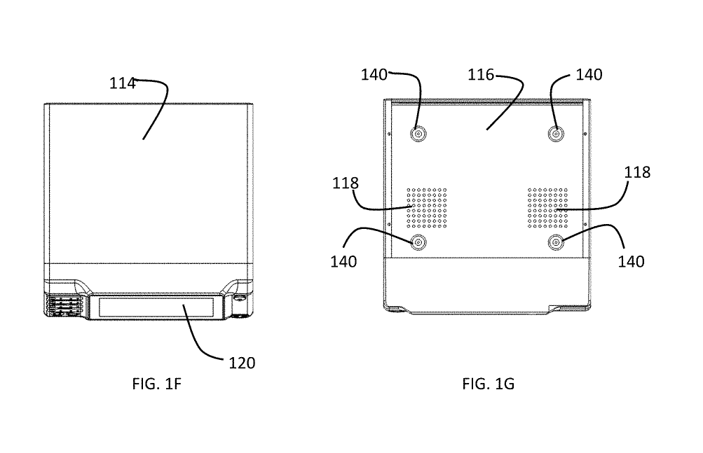

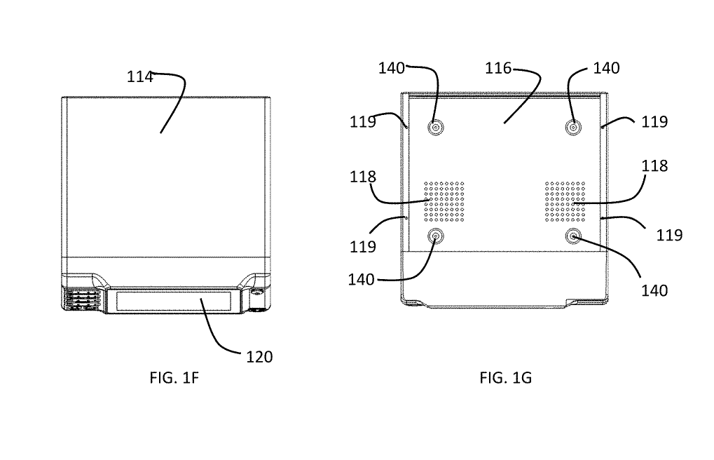

A preferred embodiment of a CAP enabled generator is described with reference to the drawings. A gas-enhanced electrosurgical generator 100 in accordance with a preferred embodiment of the present invention is shown in FIGS. 1A-1G. The gas-enhanced generator has a housing 110 made of a sturdy material such as plastic or metal similar to materials used for housings of conventional electrosurgical generators. The housing 110 has a removable cover 114. The housing 110 and cover 114 have means, such as screws 119, tongue and groove, or other structure for removably securing the cover to the housing. The cover 114 may comprise just the top of the housing or multiple sides, such as the top, right side and left side, of the housing 110. The housing 110 may have a plurality of feet or legs 140 attached to the bottom of the housing. The bottom 116 of the housing 110 may have a plurality of vents 118 for venting from the interior of the gas-enhanced generator.

On the face 112 of the housing 114 there is a touch-screen display 120 and a plurality of connectors 132, 134 for connecting various accessories to the generator, such as an argon plasma probe, a hybrid plasma probe, a cold atmospheric plasma probe, or any other electrosurgical attachment. There is a gas connector 136 for connecting, for example, a CO₂ supply for insufflating an abdomen. The face 112 of the housing 110 is at an angle other than 90 degrees with respect to the top and bottom of the housing 110 to provide for easier viewing and use of the touch screen display 120 by a user.

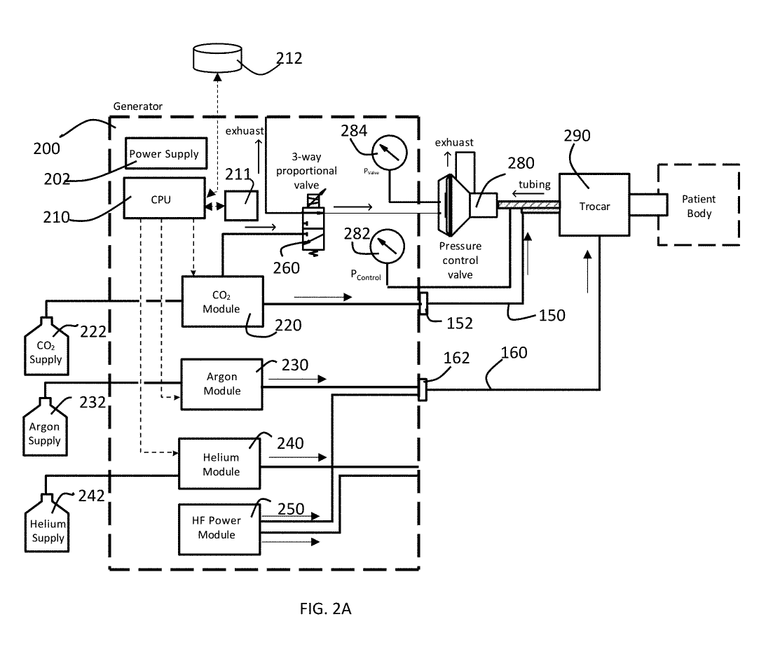

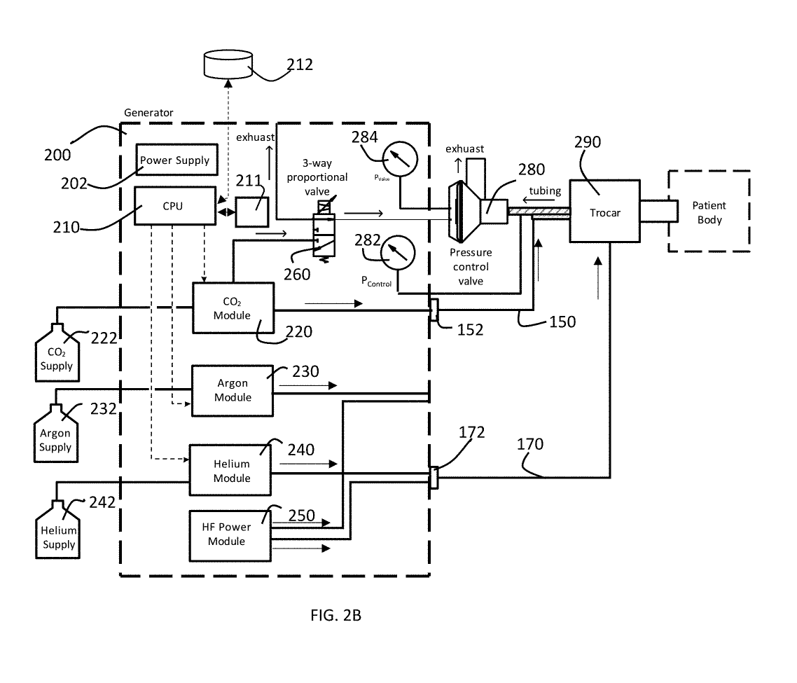

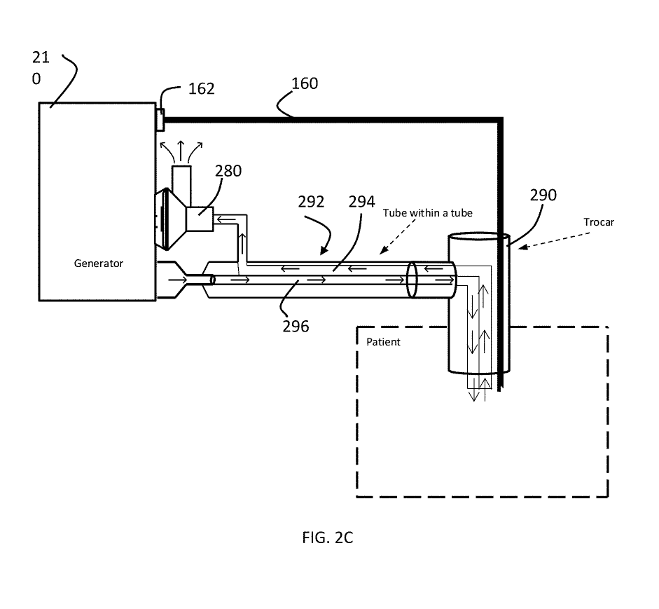

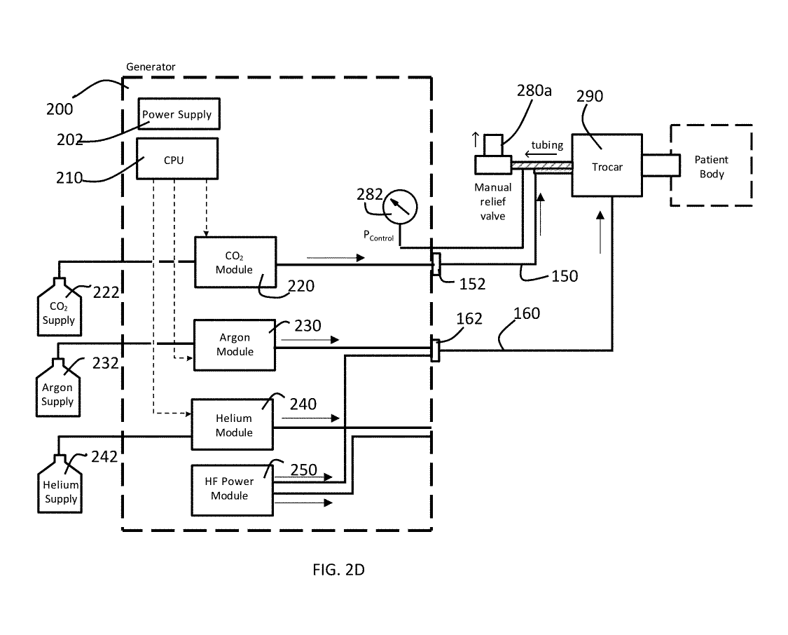

One or more of the gas control modules may be mounting within a gas-enhanced electrosurgical generator 100. A gas pressure control system 200 for controlling a plurality of gas control modules 220, 230, 240 within a gas-enhanced electrosurgical generator is described with reference to FIGS. 2A-2D. A plurality of gas supplies 222, 232, 242 are connected to the gas pressure control system 200, and more specifically, to the respective gas control modules 220, 230, 240 within the gas pressure control system 200. The gas pressure control system 200 has a power supply 202 for supplying power to the various components of the system. A CPU 210 controls the gas pressure control modules 220, 230, 240 in accordance with settings or instructions entered into the system through a graphical user interface on the display 120. The system is shown with gas control modules for CO₂, argon and helium, but the system is not limited to those particular gases. In the embodiment shown in FIGS. 2A-2D, the CO₂ is shown as the gas used to insufflate an abdomen (or other area of a patient). The gas pressure control system 200 has a 3-way proportional valve connected to the gas control module 220. While FIG.2A shows the 3-way proportional valve connected only to the CO2 control module 220, the 3-way proportional valves could be connected to a different gas control module 230 or 240. The gas pressure control system 200 further has an HF power module 250 for supplying high frequency electrical energy for various types of electrosurgical procedures. The HF power module contains conventional electronics such as are known for provide HF power in electrosurgical generators. Exemplary systems include, but are not limited to, those disclosed in U.S. Pat. Nos. 4,040,426 and 4,781,175. The system further could have a converter unit for converting the HF power to a lower frequency, such as may be used for cold atmospheric plasma and is described in U.S. Patent Application Publication No. 2015/0342663.

The outlet port of gas control module 220 is connected to a connector 136 on the generator housing. While connector 136 and the other connectors are shown on the front face of the housing 110, they could be elsewhere on the housing. The outlet ports of gas control modules 230, 240 each are connected to tubing or other channel to a connector 132. A connector 152 connects to connector 136 and is as tubing that runs to and connects to tubing 292. The tubing 292 is connected to a pressure control valve or stopcock 280 and extends into the trocar. The pressure control valve 280 is used to control pressure within the patient. The gas pressure control system further has a pressure sensor 282 connected to the tubing 292 to sense pressure in the tubing 292 and a pressure sensor 284 for sensing pressure in the pressure control valve 280. As shown in FIG. 2C, the tubing 292 is actually tube within a tube such that gas supplied from the generator travels to the trocar and patient through tube 296 and gas is released out of the patient through tube 294.

As shown in FIG. 2A the connector 132 to which control module 230 is connected has a gas-enhanced electrosurgical instrument 160 having a connector 162 connected to in. In FIG. 2A, gas control module 230 controls flow of argon gas, so the instrument 160 is an argon gas-enhanced electrosurgical tool such as an argon plasma probe such as is disclosed in U.S. Pat. No. 5,720,745, a hybrid plasma cut accessory such as is disclosed in U.S. Patent Application Publication No. 2017/0312003 or U.S. Patent Application Publication No. 2013/0296846, or a monopolar sealer such as is disclosed in U.S. Patent Application Publication No. 2016/0235462. Other types of argon surgical devices similarly can be used. As shown in FIG. 2B the connector 132 to which control module 240 is connected has a gas-enhanced electrosurgical instrument 170 having a connector 172 connected to in. In FIG. 2B, gas control module 240 controls flow of helium gas, so the instrument 170 is, for example, a cold atmospheric plasma attachment such as is disclosed in U.S. Patent Application Publication No. 2016/0095644.

The system provides for control of intraabdominal pressure in a patient. The pressure control valve 280 has a chamber within it. The pressure in that chamber is measured by pressure sensor 284. CO₂ is supplied to the chamber within pressure control valve 280 from gas control module 220 via 3-way proportional valve 260. Pressure in that chamber within the pressure control valve 280 also may be released via 3-way proportional valve 260. In this manner, the system can use the pressure sensor 284 and the 3-way proportional valve to achieve a desired pressure (set through a user interface) in the chamber within the pressure control valve 280. The pressure sensor 282 senses the pressure in the tubing 294 (and hence the intraabdominal pressure). The pressure control valve 280 then releases pressure through its exhaust to synchronize the intraabdominal pressure read by sensor 282 with the pressure in the chamber within the pressure control valve as read by pressure sensor 284. The readings from sensors 282, 284 can be provided to CPU 210, which in turn can control flow of CO₂ and one of argon and helium, depending on the procedure being performed, to achieve a stable desired intraabdominal pressure.

An alternative embodiment of the gas pressure control system is shown in FIG. 2D. This this system the automatic stopcock or pressure control valve 280 has been replaced by a manual relief valve 280a that is controlled or operated by the surgeon using the system.

A gas control module 300 in accordance with the present invention is designed for gas-enhanced electrosurgical systems. Conventionally, gas-enhanced electrosurgical systems have an electrosurgical generator and a gas control unit that have separate housings. The conventional gas control unit typically controls only a single gas such as argon, CO₂ or helium. The present invention is a gas control module 300 that may be used in a gas control unit or in a combined unit functioning both as an electrosurgical generator and as a gas control unit. Further, a plurality of gas control modules in accordance with the present invention may be combined in a single gas control unit or combination generator/gas control unit to provide control of multiple gases and provide control for multiple types of gas-enhanced surgery such as argon gas coagulation, hybrid plasma electrosurgical systems and cold atmospheric plasma systems.

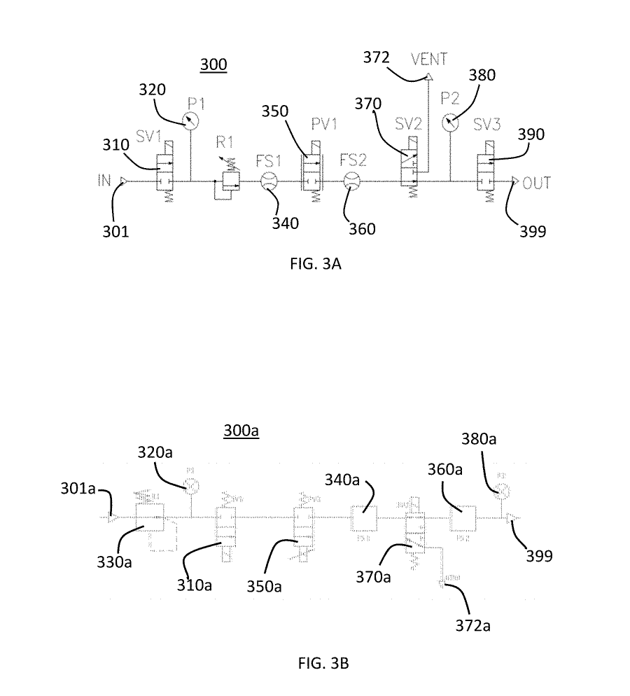

FIG. 3A is a schematic flow diagram illustrating the gas flow through the gas control module 300 and the method by which the module 300 controls the gas flow in accordance with a preferred embodiment of the present invention. As shown in FIG. 3A, the gas enters the gas control module at an inlet port (IN) 301 and proceeds to first solenoid valve (SV1) 310, which is an on/off valve. In an exemplary embodiment, the gas enters the gas module at a pressure of 75 psi. The gas then proceeds to a first pressure sensor (P1) 320, to a first pressure regulator (R1) 330. In an exemplary embodiment, the first pressure regulator (R1) 330 reduces the pressor of the gas from 75 psi to 18 psi. After the pressure regulator (R1) 330, the gas proceeds to flow sensor (FS1) 340, which sense the flow rate of the gas. Next, the gas proceeds to proportional valve (PV1) 350, which permits adjustment of a percentage of the opening in the valve. The gas then proceeds to a second flow sensor (FS2) 360, which senses the flow rate of the gas. This second flow sensor (FS2) 360 provides redundancy and thus provides greater safety and accuracy in the system. Next the gas proceeds to a second solenoid valve (SV2) 370, which is a three-way valve that provides for a vent function that can allow gas to exit the module through a vent 372. The gas then proceeds to a second pressure sensor (P2) 380, which provides a redundant pressure sensing function that against produces greater safety and accuracy of the system. Finally, the gas proceeds to a third solenoid valve (SV3) 390, which is a two-way on/off valve that is normally closed and is the final output valve in the module. The gas exits the module at and output port (OUT) 399, which is connected to tubing or other channel that provides a passageway for the gas to flow to an accessory connected to the electrosurgical unit.

FIG. 3B is a schematic flow diagram of an alternate embodiment of a gas control module illustrating the gas flow through the gas control module 300a and the method by which the module 300a controls the gas flow in accordance with a preferred embodiment of the present invention. As shown in FIG. 3B, the gas enters the gas control module at an inlet port 301a and proceeds to a first pressure regulator (R1) 330a. In an exemplary embodiment, the first pressure regulator (R1) 330a reduces the pressor of the gas from about 50-100 psi to 15-25 psi. After the pressure regulator (R1) 330a, the gas proceeds to a first pressure sensor (P1) 320a and then to a first solenoid valve (SV1) 310a, which is an on/off valve. Next, the gas proceeds to proportional valve (PV1) 350a, which permits adjustment of a percentage of the opening in the valve. Next, the gas proceeds to flow sensor (FS1) 340a, which sense the flow rate of the gas. ext the gas proceeds to a second solenoid valve (SV2) 370a, which is a three-way valve that provides for a vent function that can allow gas to exit the module through a vent 372a. The gas then proceeds to a second flow sensor (FS2) 360a, which senses the flow rate of the gas. This second flow sensor (FS2) 360a provides redundancy and thus provides greater safety and accuracy in the system. The gas then proceeds to a second pressure sensor (P2) 380a, which provides a redundant pressure sensing function that against produces greater safety and accuracy of the system. The gas exits the module at and output port 399a, which is connected to tubing or other channel that provides a passageway for the gas to flow to an accessory connected to the electrosurgical unit.

The various valves and sensors in either embodiment of the module are electrically connected to a main PCB Board through a connector 490. The PCB connector 490 is connected to a PCB Board that has a microcontroller (such as CPU 210 in the embodiment shown in FIG. 2A). As previously noted, a plurality of gas modules can be in a single gas control unit or single electrosurgical generator to provide control of multiple differing gases. The plurality of gas control modules further may be connected to the same PCB Board, thus providing common control of the modules.

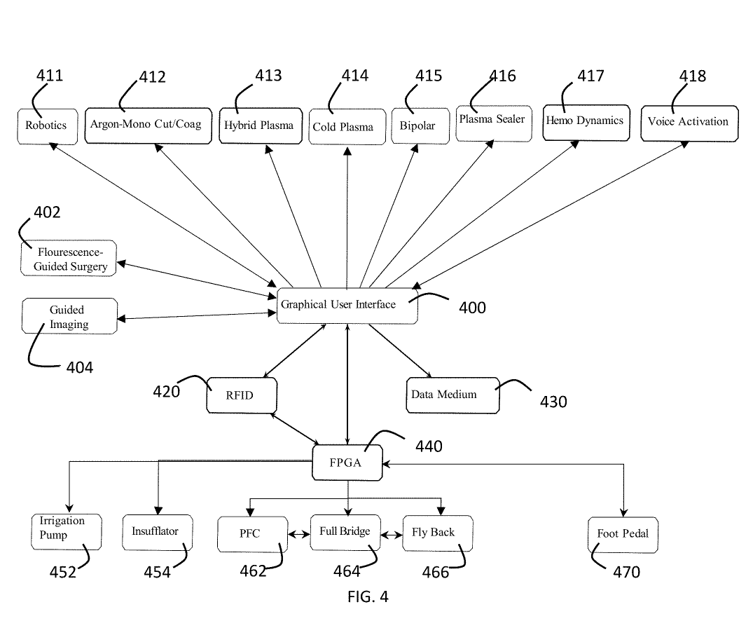

As shown in FIG. 4, the generator further may have graphical user interface 400 for controlling the components of the system using the touch screen display 120. The graphical user interface 400 for example, may control robotics 411, argon-monopolar cut/coag 412, hybrid plasma cut 413, cold atmospheric plasma 414, bipolar 415, plasma sealer 416, hemo dynamics 417 or voice activation 418. The graphical user interface further may be used with fluorescence-guided surgery 402. For example, J. Elliott, et al., “Review of fluorescence guided surgery visualization and overlay techniques,” BIOMEDICAL OPTICS EXPRESS 3765 (2015), outlines five practical suggestions for display orientation, color map, transparency/alpha function, dynamic range compression and color perception check. Another example of a discussion of fluorescence-guided surgery is K. Tipirneni, et al., “Oncologic Procedures Amenable to Fluorescence-guided Surgery,” Annals of Surgery, Vo. 266, No. 1, July 2017). The graphical user interface (GUI) further may be used with guided imaging such as CT, MRI or ultrasound. The graphical user interface may communicate with RFID 420 (such as may be found in various electrosurgical attachments) and may collect and store usage data in a storage medium 430. The graphical user interface 400 communicates with FPGA 440, which may control irrigation pump 452, insufflator 454, PFC 462, full bridge 464 for adjusting the power output, fly back 466 for regulating the power (DC to AC) and a foot pedal 470. The GUI 400 further communicates with a database of cancer cell line data with associated predicted CAP settings or dosages via the CPU 210. The databases storage may be internal memory or other internal storage 211 or external storage 212 as shown in FIGS. 2A and 2B. The data storage 430 in FIG. 4 may be in one or both of these memories or storages 211 or 212.

### Experiments:

In the following experiments, we first demonstrate that the H₂O₂ consumption rate of cancer cells after CAP treatment is a key factor determining the specific vulnerability of cancer cell lines to CAP. The higher H₂O₂ consumption rate of cancer cells during the initial 3 hours after CAP treatment, results in a less degree of cytotoxicity with CAP treatment. Cancer cells having the capacity to quickly clear the extracellular H₂O₂ are more likely to survive compared with other cells which consume the extracellular H₂O₂ more slowly.

### Methods and Materials.

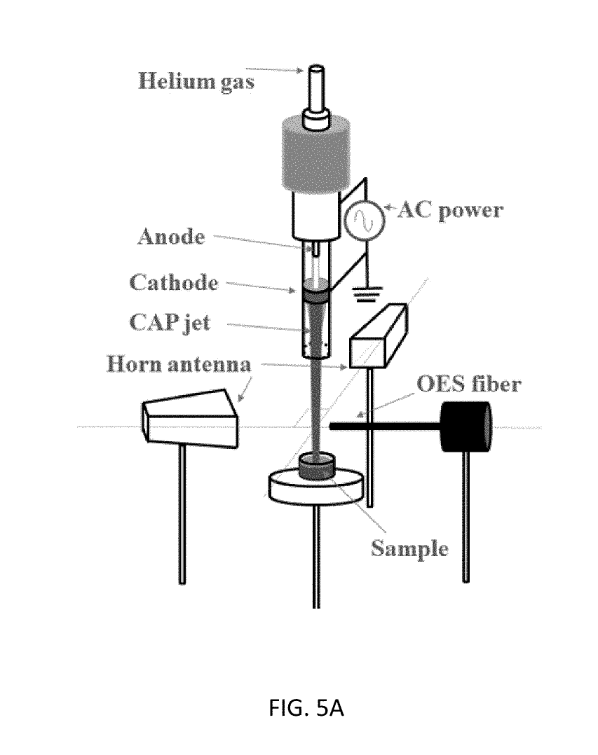

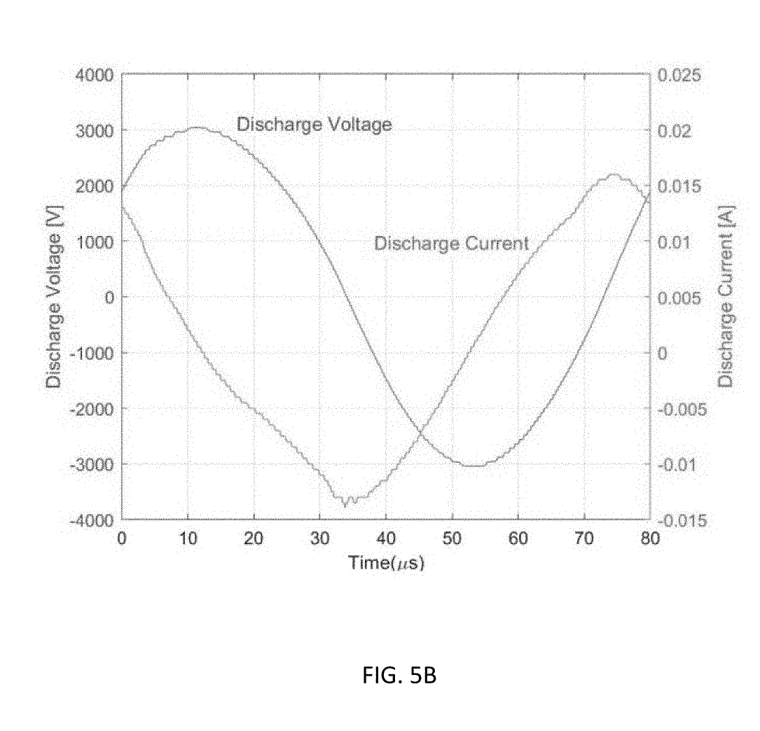

CAP device. The experimental setup is show in in FIG. 5A. The CAP device used in this study is a typical CAP jet generator using helium as the carrying gas. The detailed description of this device has been illustrated in previous studies. See, Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015); Shashurin A, Stepp M A, Hawley T S, Pal-Ghosh S, Brieda L, Bronnikov S, Jujus R A and Keidar M, “Influence of cold plasma atmospheric jet on surface integrin expression of living cells,” Plasma Process. Polym. 7 294-300 (2010). Briefly, a violet plasma jet is formed below the main discharge area between a central anode and an annular grounded cathode and flows out a glass tube with a diameter of 4.5 mm. The discharge is driven by a 12.5 kHz alternating current voltage (3 kV)(shown in FIG. 5B). The flow rate of the carrying gas is about 4.7 L/min. The optical emission spectrum (OES) of the plasma jet 20 mm to the glass tube nozzle and 5 mm to the jet axis is shown in FIG. 5C. The spectrum is a typical one of a helium CAP jet. The N₂ peaks are mainly results of the first positive system (B³Π_{g}=>A³Σᵤ⁺) and the second positive system (C³Πᵤ=>B³Π_{g}) with different vibration quantum numbers. The 706.5 nm peak from energetic triplet helium implies the existence of its metastable state 2³S which plays an important role during the jet propagation, such as the Penning ionization that He(2³S)+N₂=>He+N₂⁺+e. The 777 nm peak indicates the existence of atomic oxygen which can be a result of the dissociative attachment that e+O₂=>O+O⁻, a major process of electron density reduction.

Cell cultures. The experiment investigated 10 cancer cell lines, which include many representative cell lines in plasma medicine. Human pancreas ductal adenocarcinoma cell line (PANC-1) was purchased from American Type Culture Collection (ATCC). Other cell lines were donated by several labs at the George Washington University. These cells were all purchased from ATCC by the different labs. Human pancreatic adenocarcinoma cell line (PA-TU-8988T), human glioblastoma cell line (U87MG), as well as human lung carcinoma cell line (A549) were provided by Dr. Murad's lab. Human breast cancer cell lines (MDA-MB-231, MCF-7) were provided by Dr. Zhang's lab. Human ovarian carcinoma cell line (SK-OV-3), human ovarian carcinoma cell line (IGROV-1), human colorectal carcinoma cell line (HCT116), as well as human bone osteosarcoma cell line (U-2 OS) were provided by Dr. Zhu's lab. Murine melanoma cell line (B16F10) was provided by Dr. Sotomayor's lab. The medium used in the culture of B16F10 cells was composed of RPMI-1640 supplemented with 10% fetal bovine serum (Atlanta Biologicals, S11150) and 1% (v/v) penicillin and streptomycin solution (Life Technologies, 15140122). B16F10 cells can also be cultured in DMEM. In this study, we just used RPMI-1640 during the culture of B16F10 cells. All other cells were cultured in DMEM supplemented with 10% (v/v) fetal bovine serum and 1% (v/v) penicillin and streptomycin solution. For each experiment, 3×10³ cells were seeded per well on a 96-well plate (Falcon, 62406-081) and cultured 24 hours under standard culture conditions (a humidified, 37° C., 5% CO₂ environment) prior to CAP treatment.

CAP treatment or H₂O₂ treatment on cancer cells. Prior to CAP treatment, all medium used to culture cells overnight was removed. To perform the direct CAP treatment, the gap between the bottom of the 96-well plate and the CAP source was set to 3 cm. Subsequently, 100 μL of fresh DMEM or RPMI-1640 (only for B16F10 cells) was added to the cancer cells in the 96-well plate. The CAP jet was then used to vertically treat each well for 1 min, 2 min, or 3 min. H₂O₂-containing medium was made by adding 9.8 M H₂O₂ standard solution (216763, Sigma-Aldrich) in DMEM or RPMI-1640 (only for B16F10 cells). 100 μL of H₂O₂-containing medium was then added to the cancer cells. After direct CAP or H₂O₂ treatment, the cancer cells were cultured under the standard conditions for 3 days prior to performing the cell viability assay. In all cases, the control group consisted of cancer cells grown in fresh DMEM without CAP or H₂O₂ treatment.

Cell viability assay. MTT (3-(4,5-Dimethyl-2-thiazol)-2,5-Diphenyl-2H-tetrazolium Bromide) assay was performed following the standard protocols provided by Sigma-Aldrich. The absorbance at 570 nm was measured by a H1 microplate reader (Hybrid Technology). The measured absorbance was processed to be a relative cell viability by the division between the data of the experimental group and the control group.

Measuring the H₂O₂ consumption rate by cancer cells. The CAP-stimulated DMEM (PSM) was made by treating 8 mL DMEM in the well on a 6-well plate for 8 min. The measured H₂O₂ concentration in the CAP-treated medium was 48.8±6.5 μM. H₂O₂-containing DMEM was made as above. The same protocol was used on all cell lines. First, 100 μL of cells at a concentration of 6×10⁴ cells/mL was seeded in each well. 3 wells were used for each test. Cells were then cultured for 24 hours under standard conditions. 100 μL of sample solution was added to the wells. After that, the H₂O₂ assay was performed every hour in triplicate in the following 3 hours. In each measurement, 50 μL of medium was collected and immediately transferred to a well on a black clear bottom 96-well plate (Falcon) followed by an H₂O₂ assay.

F. Extracellular H₂O₂ Assay

The H₂O₂ concentration was measured using the Fluorimetric Hydrogen Peroxide Assay Kit (Sigma-Aldrich, MAK165-1KT) using standard protocols provided by Sigma-Aldrich. The fluorescence was measured by a H1 microplate reader (Hybrid Technology) at 540/590 nm. The final fluorescence was obtained by deducting the fluorescence of control group from the fluorescence of experimental group. The H₂O₂ concentration was obtained based on the standard curve.

### Results and Discussion.

The initial several hours are the most important stage for determining the cytotoxicity of CAP on cancer cells [10,12,18]. Our previous studies have demonstrated that key reactive species such as H₂O₂ in the medium can be completely consumed by glioblastoma cells (U*&MG) in just 3 hours. See, Yan D, Talbot A, Nourmohammadi N, Sherman J H, Cheng X and Keidar M, “Toward understanding the selective anticancer capacity of cold atmospheric plasma—a model based on aquaporins (Review),” Biointerphases. 10 040801 (2015); Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015); Yan D, Cui H, Zhu W, Nourmohammadi N, Milberg J, Zhang L G, Sherman J H and Keidar M, “The specific vulnerabilities of cancer cells to the cold atmospheric plasma-stimulated solutions,” Sci. Rep. 7 4479 (2017). Here, we comprehensively compared the H₂O₂ consumption rates of 10 cancer cell lines during their initial 3 hours cultured in the CAP-stimulated medium, which was used to quantify the ROS-scavenging ability of cancer cells. We measured the residual H₂O₂ in the medium surrounding the cells every hour after treatment for 3 hours. The relative residual H₂O₂ concentration was obtained by the division between the residual H₂O₂ concentration and the initial H₂O₂ generation in DMEM.

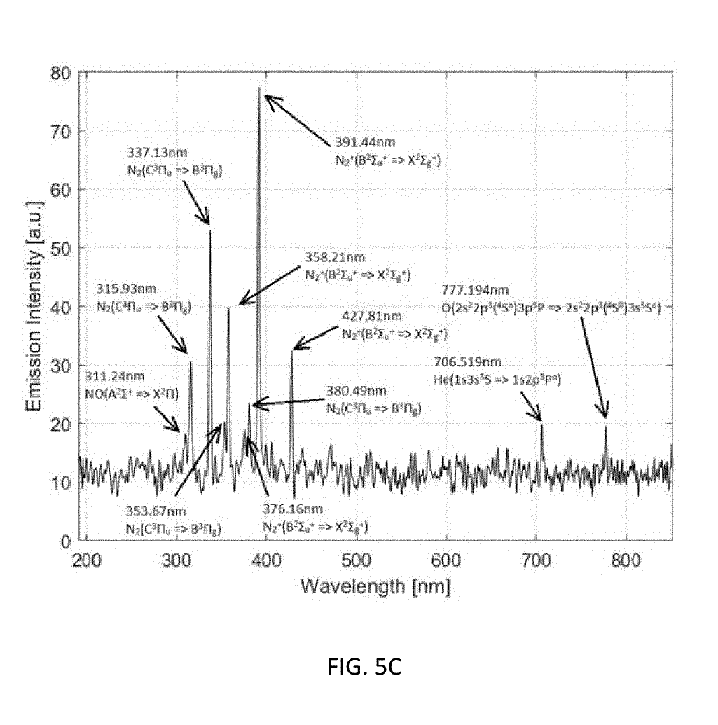

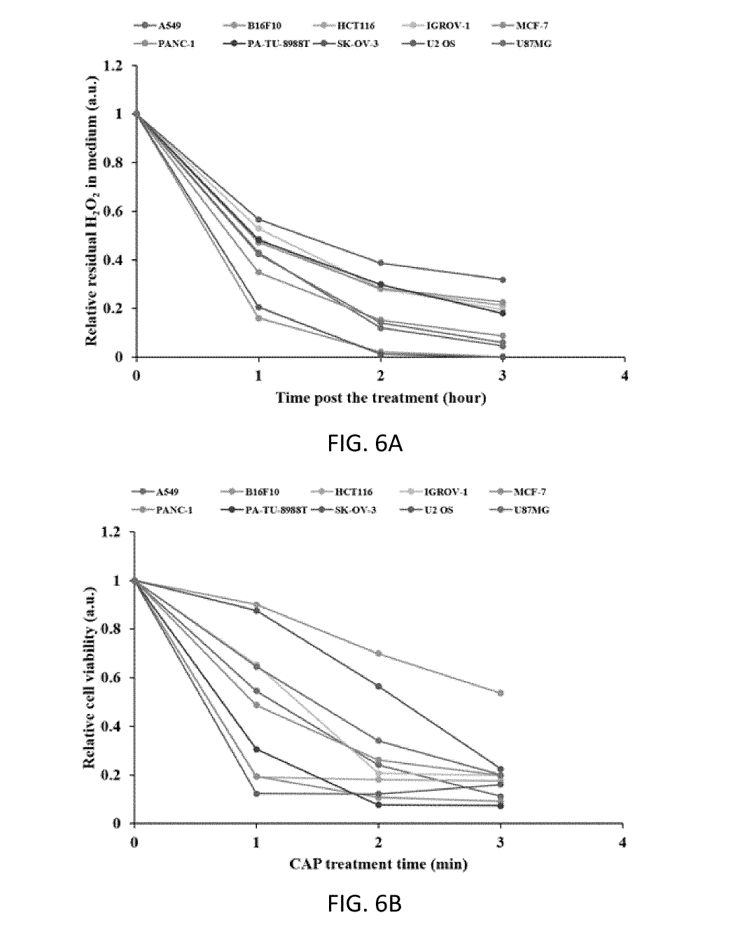

We found that the H₂O₂ consumption rate was cell specific. Among the 10 cell lines tested, B16F10 cells and SK-OV-3 cells consume H₂O₂ in the CAP treatment DMEM at the highest rate (FIG. 6A). B16F10 cells and SK-OV-3 cells clear all extracellular H₂O₂ in only 2 hours. U2 OS cells consume extracellular H₂O₂ the slowest (FIG. 6A). U2 OS cells only consume about 70% of extracellular H₂O₂ in three hours after CAP treatment. In addition, PA-TU-8998T, MCF-7 HCT116, and IGROV-1 cells have a similar but higher H₂O₂ consumption rates compared to U2 OS cells (FIG. 6A).

These 10 cancer cell lines also showed specific vulnerability to the direct CAP treatment. Due to the potential cell-based H₂O₂ generation during direct CAP treatment, the CAP device was used at a relatively low discharge voltage (3.02 kV). At such a low voltage, the cell-based H₂O₂ generation can be inhibited. See, Keidar M, Yan D, Beilis I I, Trink B and Sherman J H, “Plasmas for treating cancer: opportunities for adaptive and self-adaptive approaches,” OPINION SPECIAL ISSUE: PLASMA BIOTECHNOLOGIE, Vol. 36, Issue 6, pp. 586-593 (2018). Thus, the initial reactive species input from CAP is the same among all cell lines. Among these cell lines, B16F10 and SK-OV-3 cells are most resistant to CAP treatment (FIG. 6B). Compared with control, a treatment of B16F10 cells for 3 min with CAP led to only a 50% inhibition of cell viability. In contrast, U205, PA-TU-8998T, MCF-7, and HCT116 cells are the most vulnerable to direct CAP treatment (FIG. 5B). Clearly, these cancer cell lines have the least extracellular H₂O₂ consumption rates (FIG. 6A). The remaining cell lines generally follow this trend, in that the extracellular H₂O₂ scavenging capability of cancer cells is quasi-inversely proportional to their vulnerabilities to CAP treatment.

FIG. 6A is a graph illustrating the evolution of H₂O₂ in the extracellular environment due to the consumption of cancer cells. H₂O₂ was generated by the CAP treatment on the medium. FIG. 6B is a graph illustrating the cytotoxicity of direct CAP treatment on cancer cells grown on 96-well plates. The names of cell lines were introduced in Cell Cultures. The results are presented as the mean of three independent experiments performed in triplicate. The CAP-treated medium was made by treating 8 mL DMEM in the well on a 6-well plate for 8 min. The measured H₂O₂ concentration in the CAP-treated medium was 48.8±6.5 μM.

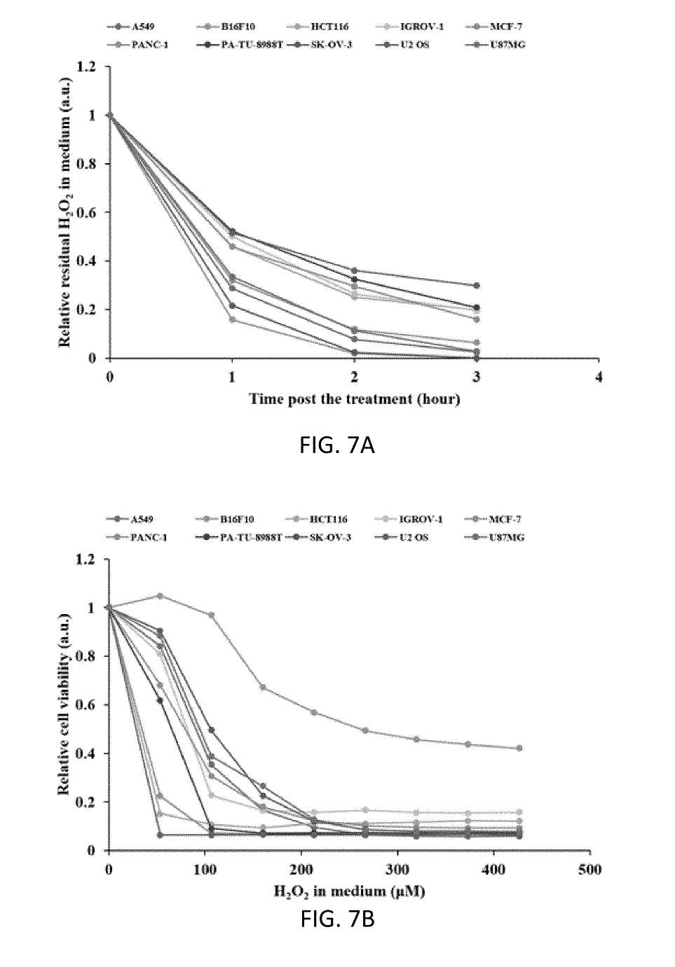

This trend was preserved when cancer cell lines were grown in the H₂O₂-containing medium. All 10 cancer cells showed nearly the same specific H₂O₂ consumption rates in the H₂O₂-containing medium as that observed in the CAP-stimulated medium (FIG. 7A). Similar, the vulnerability of cancer cells to H₂O₂ treatment is also quasi-inversely proportional to the H₂O₂ consumption rate of cancer cells (FIG. 7B). For example, B16F10 cells, simultaneously has the strongest H₂O₂-scavenging capacity and the strongest resistance to H₂O₂ treatment. As we observed in previous studies, however, CAP treatment cannot be regarded as a simple H₂O₂ treatment. Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015). B16F 10 cells are much more resistant to H₂O₂ treatment than all other cell lines including SK-OV-3 cells (FIG. 7B). Instead, the vulnerability difference between B16F10 cells and SK-OV-3 cells during H₂O₂ treatment are much larger than that observed during CAP treatment. This difference may be due to the complicated ROS and RNS components generated in CAP treatment, which will never be generated by just a H₂O₂ treatment. See, Kurake N, Tanaka H, Ishikawa K, Kondo T, Sekine M, Nakamura K, Kajiyama H, Kikkawa F, Mizuno M and Hori M, “Cell survival of glioblastoma grown in medium containing hydrogen peroxide and/or nitrite, or in plasma-activated medium.” Arch. Biochem. Biophys. 605 102-108 (2016); Girard P-M, Arbabian A, Fleury M, Bauville G, Puech V, Dutreix M and Sousa J S, “Synergistic effect of H₂O₂ and NO₂ in cell death induced by cold atmospheric He plasma,” Sci. Rep. 6 29098 (2016). Nonetheless, the same trends observed in FIGS. 6A and 6B and FIGS. 7A and 7B clearly demonstrate that the extracellular ROS-scavenging capability of cancer cells may play key a role in the specific cytotoxicity of the extracellular ROS-based treatment.

FIG. 7A is a graph illustrating the evolution of H₂O₂ in the extracellular environment due to its consumption by cancer cells. The initial H₂O₂ concentration was 53.3±9.1 μM, which was the H₂O₂ concentration generated in the 8 mL medium after 8 min of CAP treatment. FIG. 7B is a graph illustrating the cytotoxicity of H₂O₂ treatment on cancer cells. The H₂O₂ concentrations shown here are the integral multiples of 53.3 μM. The results are presented as the mean of three independent experiments performed in triplicate.

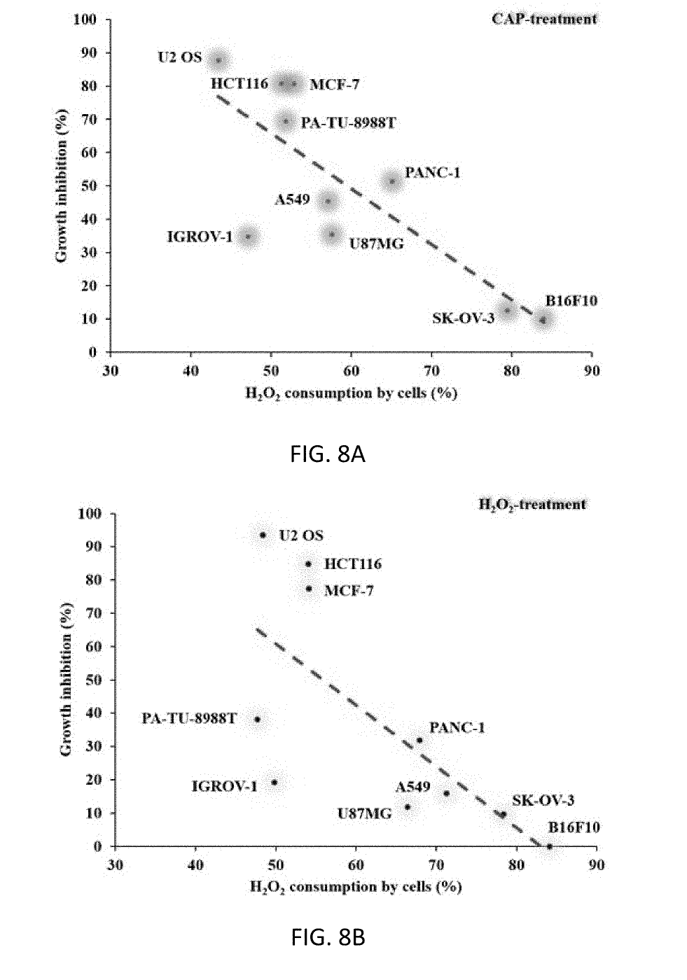

The correlation between the H₂O₂ consumption rate of cancer cells and the cytotoxicity of CAP treatment or H₂O₂ treatment on cancer cells is summarized and shown in FIGS. 8A and 8B. The inversely proportional correlation between the H₂O₂ consumption rate and the cytotoxicity of CAP is more pronounced in the case of the CAP treatment (FIG. 8A) than that of the H₂O₂ treatment (FIG. 8B). than that of the H₂O₂ treatment (FIG. 8B). Our finding provides a simple method to predict the cytotoxicity of CAP treatment on different cancer cells. This method can be easily done in any laboratories doing plasma medicine-related research. To date, this is the first attempt to connect the previous observed diverse vulnerabilities of different cancer cells to CAP treatment with a clear but easily measurable cellular function. We did not tend to use this method to replace any further biochemical analysis such as western blot and PCR. We just provide an easier method to preliminarily predict the cytotoxicity of CAP treatment, which not only provides clues to understand the anti-cancer mechanism of CAP treatment in terms of the cellular anti-oxidant function but also provides a framework to perform further studies in depth.

The H₂O₂ consumption rate of cancer cells may be the explanation at the cellular level for the correlation between the expression of p53 gene and the specific cytotoxicity of CAP treatment. p53 regulates the expression of the anti-oxidant system. See, Maillet A and Pervaiz S, “Redox regulation of p53, redox effectors regulated by p53: a subtle balance,” Antioxid. Redox Signal. 16 1285-1294 (2012). Thus, the vulnerability of cancer cells to CAP treatment may be significantly affected by the intracellular anti-oxidant system. For example, A549 and U87MG cells are known as peroxide-resistant cell lines. See, Bojes H K, Suresh P K, Mills E M, Spitz D R, Sim J E and Kehrer J P, “Bcl-2 and Bcl-X(L) in peroxide-resistant A549 and U87 mg cells,” Toxicol. Sci. 42 109-116 (1998). The overexpression of the bcl-2 and the related bcl-xL protooncogene proteins and catalase may contribute to their H₂O₂-resistant feature through inhibiting apoptosis induced by oxidants and the scavenging intracellular H₂O₂, respectively. The catalase activity is a major determinant of the cellular resistance to H₂O₂ toxicity. Spitz D R, Adams D T, Sherman C M and Roberts R J, “Mechanisms of cellular resistance to hydrogen peroxide, hyperoxia, and 4-hydroxy-2-nonenal toxicity: the significance of increased catalase activity in H₂O₂-resistant fibroblasts,” Arch. Biochem. Biophys. 292 221-227 (1992). The specific catalase expression levels in cancer cells may explain the correlation between the specific H₂O₂ consumption rate of cancer cells and the specific vulnerability of cancer cells to CAP treatment or H₂O₂ treatment. It has been observed that decreasing the expression of Cu, Zn-SOD or Mn-SOD increased the cell death of HeLa cancer cells after CAP treatment. We will systematically investigate the underlying mechanism in the further studies, which will include the potential link between the expression level of p53 and the extracellular H₂O₂ scavenging rate and the link between the expression level of anti-oxidant system such as catalase and the cytotoxicity of CAP treatment. This explanation is consistent with our previous model that catalase may play an important role in the selective anti-cancer capacity of CAP, since cancer cells tend to express less catalase compared with their corresponding homologous normal cell lines in many cases. See, Yan D, Sherman J H and Keidar M, “Cold atmospheric plasma, a novel promising anti-cancer treatment modality,” Oncotarget. 8 15977-15995 (2017).

FIGS. 8A and 8B are graphs illustrating the quasi-inversely proportional correlation between the H₂O₂ consumption rates of cancer cells and the growth inhibition effect of CAP treatment or H₂O₂ treatment on cancer cells. FIG. 8A shows CAP treatment while FIG. 8B shows H₂O₂ treatment. The red lines are the trend lines drawn by Excel 2016. The H₂O₂ consumption rate and the growth inhibition rate were calculated based on the following formulas. The H₂O₂ consumption rate (%)=100×(1−the residual H₂O₂ concentration measured at the 1^{st} hour post the treatment). The residual H₂O₂ concentration was shown in FIG. 6A, and FIG. 7A. For CAP treatment, the growth inhibition rate (%)=100×(1−relative cell viability [1 min of CAP treatment, FIG. 7B]). For H₂O₂ treatment, the growth inhibition rate (%)=100×(1−relative cell viability [53.3 μM of H₂O₂ treatment, FIG. 8B]).

### CONCLUSIONS

The H₂O₂ consumption rate of cancer cells is an important cellular physiological marker to predict the cytotoxicity of CAP treatment or H₂O₂ treatment on cancer cell lines in vitro. The cancer cells which can clear the extracellular H₂O₂ at a faster rate tend to show stronger resistance to CAP treatment or H₂O₂ treatment. This trend firstly provides a simple method to predict the vulnerability of cancer cells to CAP treatment by monitoring the evolution of H₂O₂ during the initial several hours post treatment.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for applying cold atmospheric plasma treatment to target tissue comprising:
selecting through a graphical user interface a particular cancer cell line associated with target tissue;
retrieving, with said computing device, settings data from a database of cell line data and associated settings data in a storage; and
applying, with said computing device, said retrieved settings data to a cold atmospheric plasma system.

2. A method for applying cold atmospheric plasma treatment to target tissue comprising:
generating a database of cancer cell lines and associated cold atmospheric plasma settings;
storing said database in a storage medium;
selecting through a graphical user interface on a computing device a particular cancer cell line associated with said target tissue;
retrieving, with said computing device, settings data from a database of cell line data and associated settings data in a storage; and
applying, with said computing device, said retrieved settings data to a cold atmospheric plasma system.

3. A method for applying cold atmospheric plasma treatment to target tissue according to claim 2, wherein said predicted CAP effectiveness comprises H₂O₂ consumption rate of cancer cells after CAP treatment.

4. A method for applying cold atmospheric plasma treatment to target tissue according to claim 2, wherein said cold atmospheric plasma settings in said generated database are based upon a predicted CAP effectiveness derived an H₂O₂ consumption rate of cancer cells in a particular cancer cell line after CAP treatment.

5. A method for applying cold atmospheric plasma treatment to target tissue according to claim 2, wherein said cold atmospheric plasma settings in said generated database are based upon a predicted CAP effectiveness derived predicting cytotoxicity of cold atmospheric plasma treatment on particular cancer cell lines.

6. A method for applying cold atmospheric plasma treatment to target tissue according to claim 2, wherein said step of generating a database comprises:
removing all medium used to culture a plurality of samples of a first cancer cell line;
adding DMEM or RPMI to each of said plurality of samples of said first cancel cell line;
treating each of said plurality of samples of said first cancer cell line with direct CAP treatment, wherein each of said plurality of samples of said first cancer cell line is treated using only one of a plurality of specific sets of CAP settings and wherein at least two of said plurality of samples of said first cancer cell line are treated with different specific sets of CAP settings;
adding a pre-determined amount of H₂O₂-containing medium to each of plurality of samples of said first cancer cell line;
culturing said plurality of samples of said first cancer cell line for a pre-determined period of time under pre-determined conditions;
measuring an H₂O₂ consumption rate by cancer cells in each treated sample of said first cancer cell line; and
predicting optimum CAP settings for said first cancer cell line using obtained measurements of H₂O₂ consumption rate by cancer cells in each treated sample of said first cancer cell line.
